(19) United States
(12) Patent Application Publication
(10) Pub. No.: US 2024/0315603 A1
(43) Pub. Date: Sep. 26, 2024

(54) Patient Morphology-Driven Knee Kinematics

(71) Applicant: Howmedica Osteonics Corp., Mahwah, NJ (US)

(72) Inventors: Azhar Ali, West Orange, NJ (US); llya Borukhov, Forest Hills, NY (US); Molly Walker, Midland Park, NJ (US)

(21) Appl. No.: 18/598,563

(22) Filed: Mar. 7, 2024

Related U.S. Application Data
(60) Provisional application No. 63/453,633, filed on Mar. 21, 2023.

(51) Int. Cl. A61B 5/11

(52) U.S. Cl. CPC A61B 5/1122; A61B 5/1128

(57) ABSTRACT

The present disclosure is generally directed to training and executing an artificial intelligence model to predict joint kinematics and implant alignment. Patient imaging data and associated labels, including ligament length labels related to a minimum total ligament length for a joint to move through a range of motion while maintaining a constant joint space between the bones, may be used to train the model to output predictive joint kinematics. The predictive joint kinematics and possible replacements components may be used to train another model to predict implant alignment. Implant alignment may include an orientation or alignment of the possible replacement components. During implementation, the model may receive patient specific images as input and provide, as output, an implant alignment prediction for the patient.

### CROSS REFERENCE TO RELATED APPLICATIONS

This application claims the benefit of the filing date of U.S. Provisional Patent Application No. 63/453,633 filed Mar. 21, 2023, the disclosure of which is hereby incorporated herein by reference.

### BACKGROUND

Implant design and alignment is typically determined based on the kinematics of the joint. Fluoroscopic imaging and computational modeling can be used to identify kinematics of a joint but are typically limited in sample size. Large-scale, population-based kinematics is difficult to obtain due to the significant cost in time and labor to obtain and analyze the imaging. The resulting datasets used to design implants and plan surgical interventions, therefore, tend to be small, typically less than 1,000 patients. The datasets used may not be representative of the entire patient population, or specific subsets thereof, and, therefore, may lead to sub-optimal implant design and surgical intervention choices.

### BRIEF SUMMARY

The present disclosure is generally directed to training and executing an artificial intelligence model, such as a machine learning model, to predict joint kinematics and implant alignment. The model may be trained to predict knee kinematics using patient imaging data, such as fluoroscopic images. Patient geometry, ligament attachment locations, and/or ligament kinematic contributions may be identified based on the imaging data. The imaging data may be associated with one or more labels prior to training the model. Examples of such labels may include ligament length labels related to a minimum total ligament length for a joint to move through a range of motion while maintaining a constant joint space between the bones. The patient imaging data and associated labels may be used to train the model. During training, the output of the model may be predictive joint kinematics. In examples where the joint is a knee, predictive knee kinematics may include a prediction of movement of a femur and tibia with respect to one another when the femur and tibia are healthy, or undegenerated. The output of the first machine learning model that predicts healthy kinematics from imaging data may be used to inform implant selection by a physician.

However, a second artificial intelligence model, such as a second machine learning model, may use the output of the first model to suggest one or more implants for selection by a physician, among other things. According to some examples, the output of the model may be used as input into a second machine learning model. The second model may be trained to predict implant alignment based on the predictive joint kinematics and possible implant components. Implant alignment may include, for example, a suggested implant component, and orientation of the implant component, or an alignment of the implant component. The possible implant components may be, for example, components that are available to be chosen by a physician to be implanted into a patient. For example, if the joint is a knee, the possible implant components may be femoral, tibial, and/or patellar components available to the physician. The predictive joint kinematics and possible implant components may be associated with one or more labels prior to training the second model. The predictive joint kinematics, possible implant components, and associated labels may be used to train the second model. During training, the output of the model may be a predictive implant alignment. Once the model has been trained, the model may be implemented to aid in the design of implants and planning of surgical techniques. For example, during implementation the model may receive patient image data as input into and provide, as output, an alignment prediction.

One aspect of the disclosure is directed to a method, comprising receiving as input to a first artificial intelligence model, by one or more processors, first training data including femur and tibia ligament attachment locations and femur and tibia ligament kinematic contributions, associating, by the one or more processors, at least one label of a first set of labels to respective first training data, wherein the first set of labels includes ligament length labels relating to a minimum total ligament length for a knee to move through a range of motion while maintaining a constant joint space between a femur and tibia, and training, by the one or more processors based on the first training data and the associated at least one label of the first set of labels, the first artificial intelligence model, wherein the first artificial intelligence model outputs predictive knee kinematics.

The predictive knee kinematics may include a prediction of a movement of the femur and the tibia move with respect to one another when the femur and the tibia are undegenerated. The predictive knee kinematics may correspond to a target for implanting at least one of a tibial component, a femoral component, or a patellar component. The first training data may further include tibial, femoral, and patellar geometry data.

The method may further comprise receiving as input to a second artificial intelligence model, by the one or more processors, second training data, wherein the second training data includes the predictive knee kinematics of the first machine learning model and information related to possible tibial components, femoral, and patellar components, associating, by the one or more processors, at least one label of a second set of labels to respective second training data, wherein the second set of labels includes alignment labels relating to an alignment of the possible tibial, femoral, and patellar components, training, by the one or more processors based on the second training data and the associated at least one label of the second set of labels, the second artificial intelligence model, wherein the second artificial intelligence model outputs predictive implant alignment for at least one of the possible femoral components, the possible tibial components, or the possible patellar components.

The predictive implant alignment may include at least one of a suggested femoral component, a suggested tibial component, a suggested patellar component, an orientation of the femoral component, an orientation of the tibial component, an orientation of the patellar component, or an alignment of the femoral, tibial, and patellar components. The predictive implant alignment may include at least one of a varus-valgus alignment, internal-external rotational alignment, medial-lateral position, anterior-posterior position, or flexion-extension position of the suggested femoral and tibial components. The predictive implant alignment may further include a posterior slope orientation of the suggested tibial component.

The method may further comprise executing the second artificial intelligence model to identify at least one of an implant or an alignment of an implant, the executing comprising: receiving at least one patient specific image as input into the second artificial intelligence model, and determining, using the second artificial intelligence model, an alignment prediction for the at least one patient specific image. The alignment prediction for the at least one patient specific image may include at least one of a suggested femoral component, a suggested tibial component, a suggested patellar component, an orientation of the femoral component, an orientation of the tibial component, an orientation of the patellar component, or an alignment of the femoral, tibial, and patellar components. The at least one patient specific image may include a CT scan.

The method may further comprise determining, by the one or more processors based on the at least one patient specific image, a volumetric strain metric for a ligament in the knee; and providing, as input into the second artificial intelligence model, the volumetric strain metric. Determining the volumetric strain metric may comprise comparing, by the one or more processors, a first volume of the ligament in a first pose to a second volume of the ligament in a second pose, wherein the ligament is in a taut condition in the first and second poses; and determining, by the one or more processors based on the comparison, a change in volume between the first pose and the second pose, wherein the change in volume corresponds to the volumetric strain metric.

Another aspect of the disclosure is directed to a system comprising one or more processors. The one or more processors may be configured to receive as input to a first artificial intelligence model first training data including femur and tibia ligament attachment locations and femur and tibia ligament kinematic contributions, associate at least one label of a first set of labels to respective first training data, wherein the first set of labels includes ligament length labels relating to a minimum total ligament length for a knee to move through a range of motion while maintaining a constant joint space between a femur and tibia, and train, based on the first training data and the associated at least one label of the first set of labels, the first artificial intelligence model, wherein the first artificial intelligence model outputs predictive knee kinematics.

Yet another aspect of the disclosure is directed to a computer-readable storage medium, the computer-readable storage medium including instructions that when executed by one or more processors, cause the one or more processors to receive as input to a first artificial intelligence model first training data including femur and tibia ligament attachment locations and femur and tibia ligament kinematic contributions, associate at least one label of a first set of labels to respective first training data, wherein the first set of labels includes ligament length labels relating to a minimum total ligament length for a knee to move through a range of motion while maintaining a constant joint space between a femur and tibia, and train, based on the first training data and the associated at least one label of the first set of labels, the first artificial intelligence model, wherein the first artificial intelligence model outputs predictive knee kinematics.

Another aspect of the disclosure is directed to a method, comprising receiving as input to an artificial intelligence model, by one or more processors, first training data including femur and tibia ligament attachment locations and femur and tibia ligament kinematic contributions, associating, by the one or more processors, at least one label of a first set of labels to respective first training data, wherein the first set of labels includes ligament length labels relating to a minimum total ligament length for a knee to move through a range of motion while maintaining a constant joint space between a femur and tibia, and receiving as input to the artificial intelligence model, by the one or more processors, second training data, wherein the second training data includes predictive knee kinematics and information related to possible tibial components, femoral, and patellar components, associating, by the one or more processors, at least one label of a second set of labels to respective training data, wherein the second set of labels includes alignments labels relating to an alignment of the possible tibial, femoral, and patellar components, and training, by the one or more processors based on the first and second training data, the associated at least one label of the first set of labels and the at least one label of the second set of labels, the machine learning model, wherein the artificial intelligence model outputs predictive implant alignment for at least one of the possible femoral components, the possible tibial components, or the possible patellar components.

The predictive knee kinematics may include a prediction of a movement of the femur and the tibia move with respect to one another when the femur and the tibia are undegenerated. The predictive knee kinematics may correspond to a target for implanting at least one of a tibial component, a femoral component, and a patellar component. The first training data may further include tibial, femoral, and patellar geometry data. The predictive implant alignment may include at least one of a suggested femoral component, a suggested tibial component, a suggested patellar component, an orientation of the femoral component, an orientation of the tibial component, an orientation of the patellar component, or an alignment of the suggested femoral, tibial, and patellar components. The predictive implant alignment may include at least one of a varus-valgus alignment or internal-external rotational alignment of the suggested femoral and tibial components. The predictive implant alignment may include a posterior slope orientation of the suggested tibial component.

The method may further comprise executing the artificial intelligence model to identify at least one of an implant or an alignment of an implant. The executing may comprise receiving at least one patient specific image as input into the artificial intelligence model, and determining, using the machine learning model, an alignment prediction for the at least one patient specific image. The alignment prediction for the at least one patient specific image may include at least one of a suggested femoral component, a suggested tibial component, a suggested patellar component, an orientation of the femoral component, an orientation of the tibial component, an orientation of the patellar component, or an alignment of the suggested femoral, tibial, and patellar components. The at least one patient specific image may include a CT scan.

The method may further comprise determining, by the one or more processors based on the at least one patient specific image, a volumetric strain metric for a ligament in the knee; and providing, as input into the second artificial intelligence model, the volumetric strain metric. Determining the volumetric strain metric may comprise comparing, by the one or more processors, a first volume of the ligament in a first pose to a second volume of the ligament in a second pose, wherein the ligament is in a taut condition in the first and second poses; and determining, by the one or more processors based on the comparison, a change in volume between the first pose and the second pose, wherein the change in volume corresponds to the volumetric strain metric.

Yet another aspect of the disclosure is directed to a system comprising one or more processors. The one or more processors may be configured to receive as input to an artificial intelligence model first training data including femur and tibia ligament attachment locations and femur and tibia ligament kinematic contributions, associate at least one label of a first set of labels to respective first training data, wherein the first set of labels includes ligament length labels relating to a minimum total ligament length for a knee to move through a range of motion while maintaining a constant joint space between a femur and tibia, receive as input to the artificial intelligence model second training data, wherein the second training data includes predictive knee kinematics and information related to possible tibial components, femoral components, and patellar components, associate at least one label of a second set of labels to respective training data, wherein the second set of labels includes alignments labels relating to an alignment of the possible tibial, femoral components, and patellar components, and train, based on the first and second training data, the associated at least one label of the first set of labels and the at least one label of the second set of labels, the artificial intelligence model, wherein the artificial intelligence model outputs predictive implant alignment for at least one of the possible femoral components, the possible tibial components, or the possible patellar components.

Another aspect of the disclosure is directed to a computer-readable storage medium, the computer-readable storage medium including instructions that when executed by one or more processors, cause the one or more processors to receive as input to an artificial intelligence model first training data including femur and tibia ligament attachment locations and femur and tibia ligament kinematic contributions, associate at least one label of a first set of labels to respective first training data, wherein the first set of labels includes ligament length labels relating to a minimum total ligament length for a knee to move through a range of motion while maintaining a constant joint space between a femur and tibia, receive as input to the artificial intelligence model second training data, wherein the second training data includes predictive knee kinematics and information related to possible tibial components, femoral components, and patellar component, associate at least one label of a second set of labels to respective training data, wherein the second set of labels includes alignments labels relating to an alignment of the possible tibial, femoral, and patellar components, and train, based on the first and second training data, the associated at least one label of the first set of labels and the at least one label of the second set of labels, the artificial intelligence model, wherein the artificial intelligence model outputs predictive implant alignment for at least one of the possible femoral components, the possible tibial components, or the possible patellar components.

### BRIEF DESCRIPTION OF THE DRAWINGS

FIG. 1 is a block diagram of an example system in accordance with aspects of the disclosure.

FIG. 2 is a flow diagram for an example method of training a model to predict joint kinematics in accordance with aspects of the disclosure.

FIG. 3 is example patient imaging data with a plurality of identified landmarks in accordance with aspects of the disclosure.

FIGS. 4A-4C illustrate various stages of flexion of a knee without optimized joint kinematics in accordance with aspects of the disclosure.

FIGS. 5A-5C illustrate various stages of flexion of a knee with optimized joint kinematics in accordance with aspects of the disclosure.

FIG. 6 is a flow diagram for an example method of training a model to predict implant alignment in accordance with aspects of the disclosure.

FIG. 7 is a flow diagram for an example method of training a model to predict joint kinematics and implant alignment in accordance with aspects of the disclosure.

FIG. 8 is a flow diagram for an example method of executing the artificial intelligence model(s) in accordance with aspects of the disclosure.

FIG. 9A is a flow diagram for an example method of training an artificial intelligence model to predict joint kinematics in accordance with aspects of the disclosure.

FIG. 9B is a flow diagram for an example method of training an artificial intelligence model to predict implant alignment in accordance with aspects of the disclosure.

FIG. 9C is a flow diagram for an example method of executing artificial intelligence models to predict implant alignment in accordance with aspects of the disclosure.

### DETAILED DESCRIPTION

The present disclosure is generally directed to the training and implementation of a model to predict joint kinematics and implant alignment which can be used to select/design an implant and/or preoperatively plan the alignment of an implant to return a specific patient's joint to its pre-degenerated/pre-diseased kinematics. Therefore, predictive joint kinematics, as understood herein, is the movement of bones of a joint with respect to one another through a functional range of motion when the bones are in an undegenerated state and the joint is otherwise healthy, a target for implanting replacement components, etc. Predictive implant alignment may be, for example, a suggested replacement component(s), an orientation of the replacement component, an alignment of the replacement component, etc.

According to one aspect of the present disclosure, an artificial intelligence model, such as a first machine learning (“ML”) model, may be trained using patient imaging data as training data. The patient imaging data may include, x-rays, CT scans, MRI scans, bone image data, etc. The patient imaging data may include information, such as patient geometry (e.g., three-dimensional coordinates of articular surface), ligament attachment locations, and ligament kinematic contributions. Such patient imaging data used for training the first ML model is preferably taken from a healthy, undegenerated joint and may be taken from a bone database that may be a collection of bone data from cadavers and/or living patients. The database may store a collection of images having patient imaging data. The images may be, for example, CT-scans. The collection of CT-scans or a subset of the collection of CT-scans may be used to determine bone morphology and an associated fit with replacement components, e.g., implants. The bone morphology associated with the patient imaging data may be used as input to train the first ML model. According to some examples, the database may allow the first ML model to be trained to a specific set of patient imaging data associated with a particular patient population which can be selected, for example, based on certain characteristics like race, sex, height, weight, and the like. The information obtained from the patient imaging data may be associated with a label that is used to train the first ML model. The label may be a ligament length label relating to a total ligament length for each ligament of the joint to move through a range of motion while maintaining a constant joint space. According to some examples, the total ligament length for each ligament of the joint may be determined based on the relative contribution of each ligament. In doing so, the total ligament length for all the ligaments of the joint may be minimized. During training, the output of the first ML model may be predictive joint kinematics.

A second ML model may be trained using the predictive joint kinematics of the first ML model and information related to possible replacement components as training data. The information related to possible replacement components may include implant geometries (e.g., three-dimensional coordinates of articular surface) of implants for various joints. The predictive joint kinematics and information related to possible replacement components may be associated with a label that is used to train the second ML model. The label may be an alignment label relating to an alignment of the possible replacement components to achieve the predictive joint kinematics. During training, the output of the second ML model may be predictive implant alignment. According to some examples, the predictions provided by the ML model(s) may be used to develop kinematic targets for implant designs, classify patients for selection of implant type, suggest surgical interventions, and/or establish shape-function relationships.

During implementation, the ML model(s) may receive patient imaging data as input. For example, during implementation the ML model may receive patient imaging data for a specific patient, such patient imaging data being different than the patient image data used to train the ML model. The patient imaging data may be for a given joint of a patient seeking treatment, such as a knee, elbow, wrist, shoulder, etc. The patient imaging data may include, x-rays, CT scans, MRI scans, bone image data, etc. According to some examples, when the joint of the patient is a knee, some of the patient image data may be sourced from, for example, dynamic MRI or ultrasound imaging. The MRI or ultrasound imaging may provide data associated with the ligaments of the joint. For example, the imaging may capture each of the main cruciate and collateral ligaments to present the “4-bar linkage”, e.g., the PCL, ACL, MCL, and LCL, and the patellar tendon, where each of such soft tissues are in a taut state. In some examples, the imaging may be performed down to the bundles and/or fibers within the ligaments, where the level of detail in the image may be a function of imaging resolution. The imaging may be taken in various poses, e.g., 0 degrees, 45 degrees, 60 degrees, 90 degrees, etc. of flexion, or at any other angle. The poses, including the degrees of flexion, may be determined based on a patient's maximum allowable extension limit and maximum allowable flexion limit. In some examples, some of the patient imaging data may be sourced from CT scans to provide bone data associated with the joint.

According to some examples, based on the patient image, a volumetric strain, or stiffness, metric may be determined for each ligament. For example, based on the patient image data taken from at least two different poses. The pose may include, for example, the position and orientation of the joint, ligaments within the joint, or the like. The poses may include, for example, 0 degrees, 45 degrees, 60 degrees, 90 degrees, etc. of flexion. While 0 degrees, 45 degrees, 60 degrees, and 90 degrees are provided as examples, the flexion may be at any angle throughout a range of motion. According to some examples, the poses may be patient specific, e.g., based on a patient's maximum allowable limits of extension and flexion. The ligaments within the patient image data may be segmented such that the volume of the ligament can be determined for each of at least two poses, where the ligament is in a taut condition in the poses. The volume of the ligament may correspond to the three-dimensional space occupied by the ligament. The volume of the ligament in each pose may be compared to determine a change in the volume of the ligament between at least two poses. The volumetric strain metric may be determined as a ratio of the change in volume between the two poses to the original volume.

The original volume may be, for example, the volume of the ligament transitioning from a non-taut position to a taut position as determined based on an analysis of all ligament poses captured. The volumetric strain may be determined with reference to the original volume of the ligament. In another example, the original volume may correspond to the minimum volume of the ligament for all poses. In yet another example, the original volume may correspond to the pose in which the ligament becomes taut. According to some examples, to identify the original volume, the Poisson effect of the ligament may be evaluated. For example, as the ligament begins to carry tension, e.g., when in a taut position, the cross-section of the ligament begins to decrease. The pose at which the cross-section of the ligament begins to decrease may be used to identify the transition of the ligament from a non-taut position to a taut position. The identified pose may be used to determine the original volume of the ligament.

The ML model may output a predicted implant alignment for the patient to achieve certain joint kinematics associated with such alignment. According to some examples, such joint kinematics may be those that would be best suited for the patient post-operatively. The predicted implant alignment may include a suggested replacement component, an orientation of the suggested component (e.g., internal-external rotational orientation), an alignment of the suggested component (e.g., varus-valgus alignment), a suggested cut location, orientation, and/or depth, a suggested surgical plan for implanting the suggested component, etc. According to some examples, the predicted implant alignment may include output of a predicted implant type and/or a position that most similarly reproduces the ligament strain and/or elongation pattern through a range of motion (“ROM”). The ligament strain and/or elongation pattern may be for the pre-resection and/or intact state of the joint with relatively healthy soft tissue function. In examples where the pre-resection and/or intact stat of the joint is severely diseased, the ligament strain and/or elongation pattern may be identified by the AI/ML model based on the osteophyte-free morphology of the patient. In some examples, the osteophyte-free morphology may be generated by a disease prediction model. In some examples, the ML model may account for any intra-operative ligament removal. The predicted implant alignment may optimize the position of the implant based on the patient anatomy. In some examples, the predicted implant alignment may minimize a change in ligament strain between pre-operative and intra-operative conditions. According to some examples, the predictive joint kinematics and implant alignment for the specific patient may feed back into the ML model as training data.

Training the ML model to predict joint kinematics based on patient joint geometry, ligament attachment sites, and ligament kinematic contributions may make the model more robust for predicting joint kinematics. For example, kinematics determined based on in-vitro studies that use cadaveric, multi-axis test frames to simulate loading activity kinematics with consistent loading conditions may not be applicable to all subjects and typically have a small standard size. Therefore, using training data based on patient imaging data, including patient joint geometry, ligament attachment sites, and ligament kinematic contributions may increase the robustness of predicting the joint kinematics. Further, using the predicted joint kinematics as input to a ML model to predict implant alignment may allow for greater accuracy when identifying possible replacement components, orientation of the components, and alignment of the components. For example, the ML model may be used to identify the appropriate replacement component(s) for a patient joint as well as the orientation and/or alignment of the replacement component in order to reproduce the predicted joint kinematics. This may allow users, such as physicians, to easily identify the replacement component(s) that optimizes the kinematics of the patient's joint, as compared to choosing the replacement component that matches the patient's original geometry.

Further, identifying patient-specific ligament attachment sites based on regions of highest bone density, provide an accurate and repeatable measure that can be derived from image data as compared to identifying ligament attachment sites based on observations of anatomical landmarks to estimate ligamentous attachment. Additionally, or alternatively, by optimizing the ML model to predict joint kinematics, the work performed by the joint and/or the energy expended moving the joint may be minimized by minimizing the overall ligament length of the joint throughout a range of motion. Further, the minimized ligament length and/or work expended during movement of the joint may be used to establish an objective function for the joint. According to some examples, by optimizing the ML model to predict joint kinematics, the efficiency of the joint may be restored. In some examples, the ML model may provide an objective function to maximize user effort. Further, the estimations of ligament length and/or strain, as determined by the ML model when predicting joint kinematics, may be used to determine appropriate tensions in ligament reconstruction surgeries. Additionally or alternatively, the predictive joint kinematics may be used to provide information and/or indications of possible designs of ligament grafts.

Training the ML model(s) to predict joint kinematics and/or implant alignment may provide greater accuracy when providing a suggestion of a replacement component, an orientation of the replacement component, and/or alignment of the replacement component as compared to using just the patient joint geometry. Further, using predictive motion to identify possible replacement components, as well as the alignment and orientation of the components, may provide for greater accuracy when establishing a preoperative resection plan for implantation of the suggested replacement component in the suggested alignment and orientation. Such preoperative plan may be created digitally from the suggested implant and alignment/orientation data which can then be used intraoperatively in a robotic or computer-assisted surgical procedure to execute the preoperative plan and achieve the suggested orientation/alignment for the suggested implant. An exemplary system and method for executing a preoperative plan can be found in U.S. Pat. No. 10,357,315, the disclosure of which is incorporated by reference herein in its entirety.

The ability to more accurately to predict joint kinematics and/or implant alignment using AI models increases the computational efficiency of the system by reducing the number of inputs, e.g., trial and error, required to identify a replacement component, an orientation of the replacement component, and/or alignment of the replacement component as compared to using just the patient joint geometry. Receiving less inputs to try various replacement components in various orientations and/or alignments reduces network and processor overhead associated with identifying a replacement component, an orientation of the replacement component, and/or alignment of the replacement component.

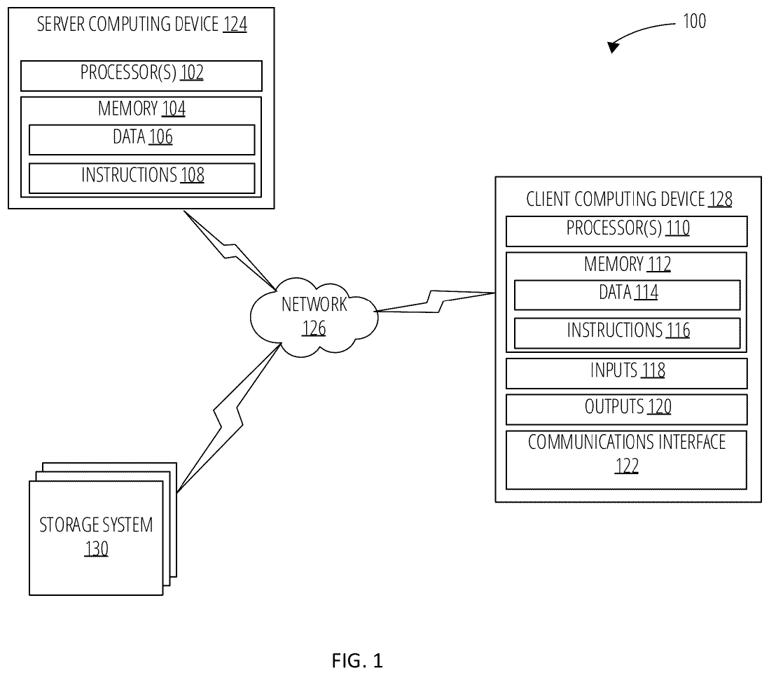

FIG. 1 illustrates an example system for predicting joint kinematics and implant alignment. As an exemplary system, it should not be considered as limiting the scope of the disclosure or usefulness of the features described herein. As shown, system can include server computing device 124, client computing device 128, and storage system 130.

The client computing device 128 may be a full-sized personal computing device and/or a mobile computing device capable of wirelessly exchanging data with a server over a network, such as the Internet. For example, client computing device 128 may be a mobile phone or device, such as smartphone, wireless-enabled PDA, a tablet PC, laptop, etc. According to some examples, client computing device 128 may be a computer or computing device coupled to a patient imaging device, such as an x-ray machine, magnetic resonance imaging (“MRI”) machine, computerized tomography (“CT”) scanner, etc.

Client computing device 128 may include one or more processors 110, memory 112, data 114, instructions 116, inputs 118, outputs 120, communications interface 122, and other components typically present in general purpose computing devices. Memory 112 of client computing device 128 can store information accessible by the one or more processors 110, including instructions 116 that can be executed by the processors 110.

Memory can also include data 114 that can be retrieved, manipulated, or stored by the processor 110. The memory 112 can be of any non-transitory type capable of storing information accessible by the processor, such as a hard-drive, memory card, ROM, RAM, DVD, CD-ROM, write-capable, and read-only memories.

The instructions 116 can be any set of instructions to be executed directly, such as machine code, or indirectly, such as scripts, by the one or more processors 110. In that regard, the terms “instructions,” “application,” “steps,” and “programs” can be used interchangeably herein. The instructions can be stored in object code format for direct processing by a processor, or in any other computing device language including scripts or collections of independent source code modules that are interpreted on demand or compiled in advance. Functions, methods, and routines of the instructions are explained in more detail below.

Data 114 may be retrieved, stored, or modified by the one or more processors 110 in accordance with the instructions 116. For instance, although the subject matter described herein is not limited by any particular data structure, the data can be stored in computer registers, in a relational database as a table having many different fields and records, or XML documents. The data can also be formatted in any computing device-readable format such as, but not limited to, binary values, ASCII or Unicode. Moreover, the data can comprise any information sufficient to identify the relevant information, such as numbers, descriptive text, proprietary codes, pointers, references to data stored in other memories such as at other network locations, or information that is used by a function to calculate the relevant data.

The one or more processors 110 can be any conventional processors, such as a commercially available CPU. Alternatively, the processors can be dedicated components such as an application specific integrated circuit (“ASIC”) or other hardware-based processor.

Although FIG. 1 functionally illustrates the processor 110, memory 112, and other elements of client computing device 128 as being within the same block, the processor, computer, computing device, or memory can comprise multiple processors, computers, computing devices, or memories that may or may not be stored within the same physical housing. For example, the memory can be a hard drive or other storage media located in housings different from that of the client computing device 128. Accordingly, references to a processor, computer, computing device, or memory will be understood to include references to a collection of processors, computers, computing devices, or memories that may or may not operate in parallel.

The inputs 118 may be, for example, a touchscreen, keyboard, mouse, microphone, etc. The inputs 118 may receive user interaction with patient imaging data. In some examples, the inputs 118 may receive user interaction with possible implant components.

The outputs 120 may include, for example, a display, such as a monitor having a screen, a projector, a television, etc. that is capable of providing a visual output to a user. The visual output may electronically display information to a user via a user interface, such as a graphical user interface. For example, the displays may electronically display the predictive joint kinematics and/or the predicted implant alignment. In some examples, the outputs 120 may include audio outputs, such as speakers, which are capable of providing an audible output to the user.

Although only one client computing device 128 is shown in FIG. 1, it should be appreciated that the system 100 may include any number of client computing devices 128, with each being at a different node of network 126. For example, a client computing device may be at a location where the patient imaging data is captured while another client computing device is at a physician's office being used to predict joint kinematics and implant alignment.

The client computing devices 128 and server computing device 124 can be at different nodes of a network 126 and capable of directly and indirectly communicating with other nodes of network 126. Although only a few computing devices are depicted in FIG. 1, it should be appreciated that a typical system can include many connected computing devices, with each different computing device being at a different node of the network 126. The network 126 and intervening nodes described herein can be interconnected using various protocols and systems, such that the network can be part of the Internet, World Wide Web, specific intranets, wide area networks, or local networks. The network can utilize standard communications protocols, such as Ethernet, WiFi and HTTP, protocols that are proprietary to one or more companies, and various combinations of the foregoing. Although certain advantages are obtained when information is transmitted or received as noted above, other aspects of the subject matter described herein are not limited to any particular manner of transmission of information.

As an example, server computing device 124 and client computing device 128 may include a communications interface 122, or web servers, capable of communicating with storage system 130, as well as other computing devices via network 126. For example, one or more server computing devices 124 may use network 126 to transmit and present information to a user on an output 120 of the client computing device 128.

The system 100 may include one or more server computing devices 124. Server computing device 124 may include processors 102, memory 104, data 106, and instructions 108 that operate in the same or similar fashion the processors 110, memory 112, data 114, and instructions 116 of client computing device 128. According to some examples, server computing device 124 may operate as a load-balanced server farm, distributed system, etc.

Although some functions described below are indicated as taking place on a single computing device having a single processor, various aspects of the subject matter described herein can be implemented by a plurality of computing devices, for example, communicating information over network 126.

Storage system 130 can be any type of computerized storage capable of storing information accessible by the server computing device 124 and/or client computing device 128, such as a hard-drive, memory card, ROM, RAM, DVD, CD-ROM, write-capable, and read-only memories. In addition, storage system 130 may include a distributed storage system where data is stored on a plurality of different storage devices which may be physically located at the same or different geographic locations. Storage system 130 may be connected to server computing device 124 and client computing device 128 via network 126 as shown in FIG. 1 and/or may be directly connected thereto (not shown). According to some examples, storage system 130 may store patient imaging data of various joints, such as CT scans, MRI scans, etc. The patient imaging data may be for healthy, undegenerated joints and/or unhealthy, degenerated joints. The storage system 130 may also store information related to implant components than can be used during a surgical procedure. The implant components may include a tibial component, a femoral component, and/or a patellar component for a knee, a glenoid component for a shoulder, a stem, ball, and socket for a hip, the stems and hinge for an elbow, prosthetics for an ankle, etc. The patient imaging data and/or the implant component information stored in storage system 130 may be used as training data for training the model to predict joint kinematics and/or implant alignment.

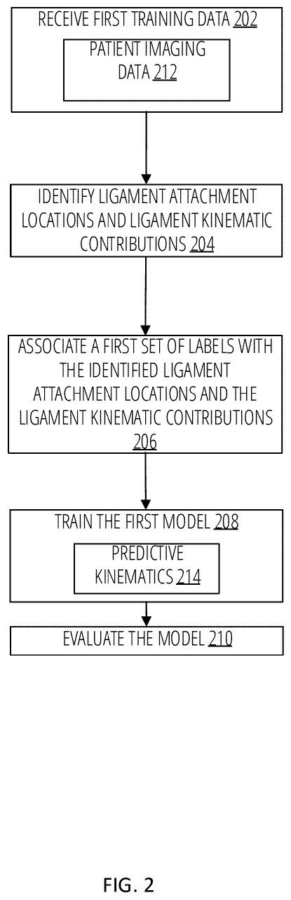

FIG. 2 is an example method for training an artificial intelligence (“AI”) model, such as a machine learning (“ML”) model. Training the model may include receiving first training data 202, identifying ligament attachment location and ligament kinematic contributions 204, associating associate a first set of labels 206 with the ligament attachment location and ligament kinematic contributions, training the first model 208, and evaluating the model 210.

Receiving the first training data 202 for the model may include obtaining patient imaging data 212. The patient imaging data may be x-ray images, CT scans, MRI scans, etc. The patient imaging data 212 may be images, or scans, of specific joints in the human body. For example, the patient imaging data 212 may be images, or scans, of a knee, elbow, shoulder, wrist, etc. The images may include bone structures, soft tissue such as ligaments and tendons, among other anatomy. The patient imaging data 212 may be images that were previously obtained and stored in storage system 130. Such storage system 130 may be a database with a collection of images, for example.

According to some examples, the training data 202 may be built based on patient imaging data. The training data 202, in conjunction with a first AI model, e.g., the first ML model, may be used to determine a minimum number of images and/or frames to analyze for each ligament. For example, the training data 202 may be evaluated using a dimensionality reduction Al and/or ML algorithm. The AI model may be trained to evaluate which input variables have the most impact such that subsequent models can be executed with a reduced number of input variables. As an example, the AI model may be a principal component analysis (“PCA”) model. The PCA model can be used to analyze all kinematic poses and determine the flexion angles that are most influential, or impactful, to the output prediction of the AI model.

In some examples, the training data 202, in conjunction with the first AI model, may be used to determine a relationship between CT imaging and dynamic MRI and/or ultrasound imaging. According to some examples, the training data 202 may consist of a plurality of imaging modalities, such as CT, MRI, ultrasound, etc. One or more additional AI models may be trained an executed to identify relationships between the imaging modalities. For example, the additional AI models may be trained to predict cartilage and ligament attachments using CT images, thereby avoiding the need to obtain an MRI. According to some examples, a plurality of imaging modalities, such as CT, x-ray, MRI, ultrasound, etc., along with dynamic MRI and ligament characterization may be used as training data for the additional AI model(s). The ligament characterization may include, for example, the strain and/or elongation pattern through the ROM. The Al model(s) maybe trained to determine a prediction of the ligament characterization based on the imaging modality. For example, the AI model(s) may be trained to predict the characterization based on CT, x-ray, or other imaging modality. In execution, the AI model(s) may receive patient images from a given imaging modality. The AI model(s), when executed, may provide, as output, a ligament characterization for the joint based on the imaging modality of the patient images. By being able to predict ligament characterizations for a given imaging modality, obtaining images of the joint in multiple modalities, e.g., CT and MRI, may be negated.

Ligament attachment location and ligament kinematic contributions 204 may be identified based on the patient imaging data 212. Using the knee joint as an example, ligament attachment sights may include, for example, locations on the femur and tibia where the ligaments for the knee are attached. In some examples, the ligament attachment sites may be within a ligament attachment zones. The zone may be identified for the cruciate and collateral ligaments, such as the medial collateral ligament (“MCL”), lateral collateral ligament (“LCL”), anterior cruciate ligament (“ACL”), and posterior cruciate ligament (“PCL”). The ligament attachment zones may be, for example, spherical zones. In one example, the attachments zones may be 10-15 mm spheres. However, a spherical zone is just one example of the attachment zone shape and a size of 10-15 mm is just one example of the attachment zone size. For example, based on the joint, the shape and/or size may change. Accordingly, a spherical attachment zone having a size of 10-15 mm is just one example and is not intended to be limiting. According to some examples, the centroid with the highest density (greater than 90% threshold) may be identified to for each attachment zone to establish patient specific ligament attachment sites. Density may be measured with reference to the medical image and in terms of Hounsfield Units (“HU”), for example. Each ligament provides its own contribution to the overall kinematics of a particular joint. For example, through a range of motion of a joint, each ligament provides a resistive force which, when combined with the other ligaments, helps determine how the joint moves through the range of motion. In this regard, a ligament contribution can include force vector magnitudes and orientations which can both be determined by analyzing the bone density at the ligament attachment points. A ligament that has a greater contribution than another ligament will generally have a higher density at its attachment point in accordance with Wolff's law. Bone density at any location may be compared to reference bone density, such as from a population of bones taken from a bone database, and/or relative to other locations of the bone to normalize and determine the magnitude of the contribution. Bone density data may also be analyzed to determine the direction/orientation of the force vector. For example, multiple layers or “slices” of bone density data at a ligament attachment point may be analyzed to determine the direction of the vector passes through the points of maximum density. Exemplary methods of bone density and vector analysis of joint ligaments are described in further detail in U.S. Pat. No. 10,827,971, which is incorporated by reference herein in its entirety.

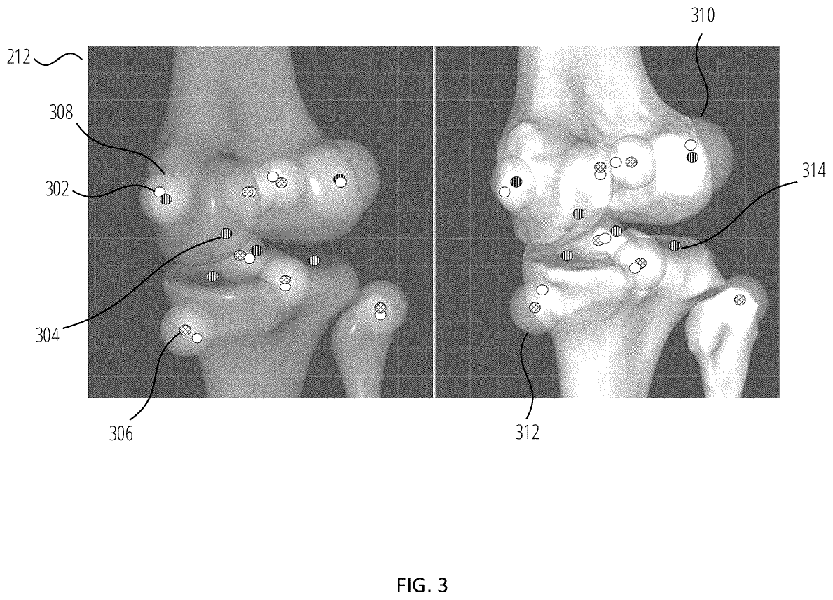

FIG. 3 illustrates an example of patient imaging data 212 of a knee joint. As shown in FIG. 3, a plurality of attachment zones 308, 310, 312 have been identified as well as a plurality of attachment sites 302, 304, 306. For purposes of clarity, only a selection of attachment zones and attachment sites have been identified using reference numbers.

The ligament attachment zones 308, 310, 312 may be identified for the ligaments in the joint. As shown, the ligament attachment zones may be identified for the cruciates and collaterals, e.g., the MCL, LCL, ACL, and PCL, of the knee. The centroid with the highest density may be identified for each zone. The highest density may be, for example, a density greater than 90% of the threshold. In some examples, the highest density may be a density greater than 50% of the threshold. Accordingly, the centroid may be determined based on a predefined percentage of a threshold density.

The attachment sites 302, 304, 306 may be identified based on anatomical features 304, statistically-generated average locations of a ligament origin and insertions 306, or patient-specific regions of highest bone density 302 within attachment zones 308, 310, 312. For example, attachment site 304, and similar attachment sites identified with the vertical line pattern, may be identified based on anatomical features, such as the epicondyles (medial and/or lateral), tuberosities, and any other anatomical feature commonly associated with ligament attachment. In another example, attachment site 306, and similar attachment sites identified with the cross-hatch pattern, may correspond to a statistically-generated average location of ligament origin and insertion. More specifically, attachment site 306, and similar cross-hatched attachment sites, may correspond to average ligament attachment locations in a joint which can be determined through a statistical analysis of a bone database and then applied to the patient's bone data 212. In a further example, attachment site 302, and similar attachments sites identified with no pattern, may correspond to a patient-specific attachment site as determined using a density-based technique. More specifically, and according to some examples, attachment sites 304 and/or 306 may be used to establish a centroid, or center, of an attachment zone 308, 310, 312 that expands outward from the centroid 304/306 and which can be explored to identify regions of highest density within zone 308, 310, 312 to mark the patient-specific ligament attachment locations. In other words, the region with the highest bone density 302 within the attachment zone 308, 310, 312 represents a patient-specific attachment location. The ligament attachment zone may, in some examples, have a radius of 10-15 mm, but may be larger or smaller depending on the joint. According to some examples, in clinical applications, such as robotic surgery, a user, such as a surgeon, may digitize the ligament attachment location as part of the clinical workflow.

Referring back to FIG. 2, training the model may include associating labels 206 with the identified ligament attachment location and ligament kinematic contributions. For example, a reviewer may attach a first set of labels to the patient imaging data 212. The labels may include, for example, applicable anatomical information and are associated with the patient imaging data from which the labels are derived. The reviewer may, additionally or alternatively, attach the first set of labels to the identified ligament attachment location and ligament kinematic contributions. For each patient image, identified ligament attachment location, and/or ligament kinematic contribution may be associated with at least one label of the first set of labels. The first set of labels may include ligament length labels. The ligament length labels may relate to a total ligament length(s) for a given joint to move through a range of motion while maintaining a constant joint space between the bones of the joint. Taking the knee as an example, the first set of labels may include a ligament length label relating to the minimal total ligament length for the knee to move through a range of motion while maintaining a constant joint space between the femur and tibia. According to some examples, the joint space may change as a function of flexion based on a set of imaging data such as x-rays, CT or static and dynamic MRI taken at multiple kinematic poses.

In some examples, the patient imaging data, ligament attachment locations and/or ligament kinematic contributions may be associated with the first labels without human review or intervention. For example, the patient imaging data, ligament attachment locations and/or ligament kinematic contributions may be automatically associated with a first label. In some examples, the patient imaging data, ligament attachment locations and/or ligament kinematic contributions may already include associated first labels at the time the first training data is received. In examples where the patient imaging data, ligament attachment locations and/or ligament kinematic contributions already include associated first labels, associating the labels with the identified ligament attachment location and ligament kinematic contributions 204 may include confirming the associated labels and/or associating additional labels with the patient imaging data, ligament attachment locations and/or ligament kinematic contributions.

At block 208, the model may be trained. The ML model may be trained using the first training data 202 and associated labels 206 to provide predictive joint kinematics based on the respective ligament length labels. The ML model may be trained to predict joint kinematics using a rule-based optimization. According to some examples, optimizing the kinematics of a joint may include maintaining a bone-to-bone distance between the bones of the joint equal to the scanned distance between the lowest points of the bones of the joint. Optimizing the kinematics of a joint may minimize the total ligament length.

According to some examples, predicting joint kinematics using rule-based optimization may include applying a weight factor to each ligament in the joint. The weight for each ligament may be the same or different. In some examples, the weight may be the same for some of the ligaments while the weight may be different for the others. As an example, the knee may include the MCL, LCL, ACL, and PCL. Each ligament may be assigned a weight. For example, the weights may be as assigned as follows: MCL_w=1; LCL_w=1; ACL_w=1−(flexion/max flexion); and PCL_w=(flexion/max flexion). The weight assigned to the ACL may decrease as the flexion of the knee increases whereas the weight assigned to the PCL may increase as the flexion of the knee increase. The rule-based optimization may, therefore, change based on the ligament attachments and weights assigned to each ligament. Accordingly, while weights between zero and one are referenced, any values may be assigned as the weight for each ligament. Further, while the different ligaments for the knee are discussed, the weights may be applied to the ligaments relative to the joint, e.g., elbow, ankle, shoulder, etc. Accordingly, the weights and ligaments provided are just one example and are not intended to be limiting.

Using the knee as an example, knee flexion may be simulated with the femur in response to the tibia local coordinate system. For example, the knee flexion may be between 0-130 degrees and may be performed in a predetermined increment, such as 5 degrees. The ML model may optimize the relationship between the tibia and femur so that the ligaments remain as short as possible at each increment and the bone-to-bone distance equals to the as-scanned distance between the lowest femoral condylar points and the tibial plateau. According to some examples, this distance may be, for example, approximately 4 mm. In some examples, the distance may be greater or less than 4 mm. Thus, the ML model may be trained to optimize the positioning of the tibial relative to the femur at each increment. Such positioning may include relative internal-external rotation, varus-valgus alignment, and the anterior-posterior and medial-lateral relationships of the distal-most contact points of the tibiofemoral condyles. The relationship of the bones at each increment when taken together form the kinematic profile of the tibiofemoral joint. Optimizing the kinematics of the various ligaments may minimize the total ligament length for the joint. According to some examples, minimization of the total ligament length is based on minimizing work, or energy, expended by the joint (e.g., the ligaments) while the knee is moving. Flexion kinematics may be imposed while the remaining 5 degrees of freedom of the knee are optimized to achieve cartilage contact and minimized ligament length.

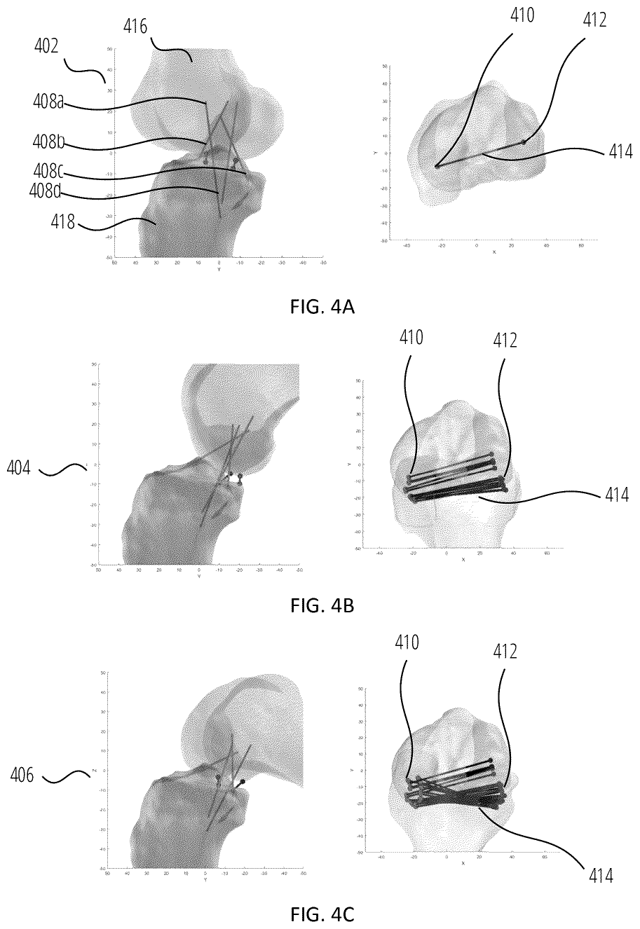

FIGS. 4A-C illustrate various stages of flexion of a knee without optimized joint kinematics. For example, images 402 illustrate a medial view (left image) and a superior-to-inferior view (right image) of a knee joint in extension, images 404 illustrate the medial and posterior view of the knee joint in partial flexion, and images 406 illustrate the medial and posterior view of the knee joint in full flexion.

The medial view of the images 402, 404, 406 include lines 408a-408d representing the ligaments of the knee joint. As the knee joint moves from extension, FIG. 4A, to flexion, FIG. 4C, the length of the ligaments 408a-408d may change. For example, some of the ligaments 408a-408d may be stretched while others return to a more relaxed, unstretched, state.

The superior-to-inferior view of the images 402, 404, 406 includes an indication of the most distal medial point 410 and lateral point 412 of the femur 416 as they contact the tibia through a range of motion. The medial point 410 represents the contact point between the medial femoral and tibial condyles and the lateral point 412 represents the contact point between the lateral femoral and tibial condyles as the femur and tibia move with respect to one another. The line 414 between the lateral point 412 and medial point 410 provides an indication of which lateral point 412 corresponds to which medial point 410 during flexion of the knee joint and also the distance between each point.

As shown in FIGS. 4A-4C, both the medial point 410 and lateral point 412 move as the knee rotates through a range of motion. Typically, in a healthy, undegenerated, optimized knee joint, the medial point 410 remains substantially stationary while the lateral point 412 translates in an anterior-posterior direction thereby providing the characteristic medial-pivot movement of a typical knee joint. However, in an unhealthy, degenerated, unoptimized joint in which ligament lengths are not constrained to a minimum, as shown in FIGS. 4A-4C, the medial and lateral points 410, 412 translate together posteriorly in a chaotic manner and to a point of near dislocation. This can cause additional, or long term, problems with the joint, such as further degeneration, patient pain, etc.

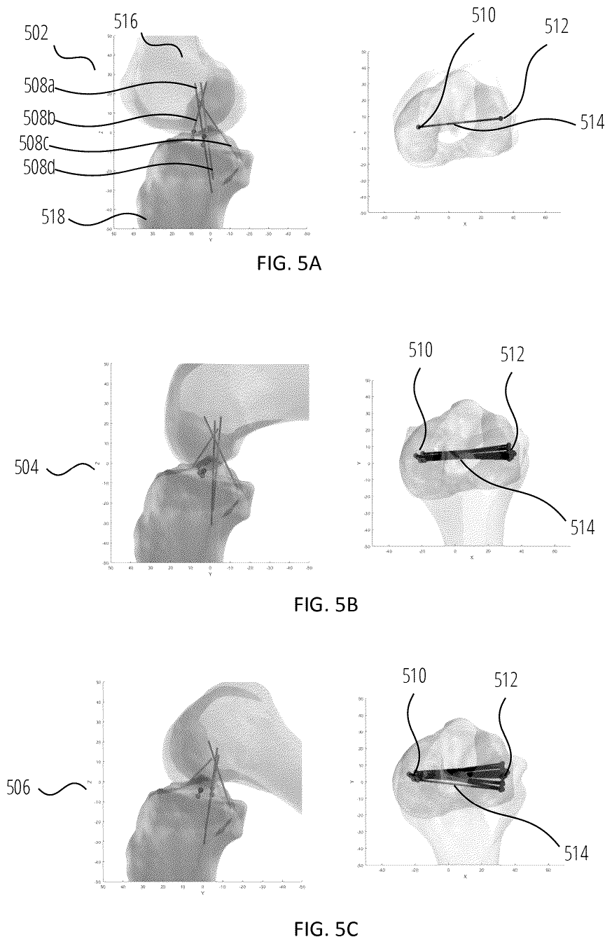

In comparison to FIGS. 4A-C, FIGS. 5A-C illustrate various stages of flexion of a knee with optimized joint kinematics. Images 502, 504, 506 illustrate a medial view (left images) and superior-to-inferior view (right images) of the knee joint in extension, partial flexion, and full flexion, respectively. Similar to FIGS. 4A-4C, lines 508a-508d represent the ligaments of the knee joint, medial point 510 represents the contact point between the medial femoral and tibial condyles, and lateral point 512 represents the contact point between the lateral femoral and tibial condyles.

As shown in FIGS. 5A-5C, the medial points 510 are substantially grouped together. This indicates that the contact point between the medial femoral and tibial condyle remains substantially stationary while the lateral contact point translates. The translation of the lateral contact point is illustrated by the movement of the lateral point 512 in FIGS. 5A-5C.

The ML model may be trained to provide optimized kinematics. For example, the predictive joint kinematics output by the ML model may be optimized using rule-based optimization. The predictive joint kinematics may, therefore, optimize the kinematics of the joint such that the predictive kinematics, or movement, of the joint reflects the typical kinematic movement of a healthy, undegenerated joint. In the example of a knee, the optimized kinematics may reflect the typical kinematic medial pivot movement for a healthy, undegenerated knee.

Referring back to FIG. 2, the model can repeat the training 208 multiple times, until meeting one or more stopping criteria, such as a maximum number of training iterations. In some examples, the stopping criteria may define a minimum improvement between training iterations. Other stopping criteria may, in some examples, be based on computing resources allocated for training, a total amount of training time, etc.

The ML model may be evaluated 210. The ML model may be evaluated based on the type of joint, the ligament attachment locations, the ligament contributions, patient geometry, etc. For example, if the ML model performed poorly in predicting kinematics for a specific type of joint, ligament attachment location, and/or ligament kinematic contribution, the ligament attachment locations and/or ligament kinematic contributions may be adjusted to create more effective training data. In some examples, the ML model may be evaluated 210 based on patient imaging data identified as corresponding to healthy subjects. Healthy subjects may include, for example, joints of patients that are undegenerated. The patient imaging data for the healthy subjects may include an indication of measure kinematics for joint extension activity using a dynamic bi-plane fluoroscopic system. For example, if the joint is a knee, the patient imaging data for healthy subjects may include an indication of measured tibiofemoral kinematics for a passive knee extension. The indication of the measurements on the patient imaging data for healthy subjects may be compared to the predictive joint kinematics of the ML model.

According to some examples, the predictive joint kinematics 214 may be stored in storage system 130 to be accessed by the first model 208 or another ML model.

According to some examples, the predictive joint kinematics 214 represents the healthy, or ideal, motion according to a patient's anatomy. The predictive joint kinematics 214 may, therefore, take into account all the ligaments of the given joint. However, during certain joint replacements, such as a knee replacement, certain ligaments, such as the ACL and/or PCL, may be removed when replacing the joint. The function of the removed ligaments may be included in the predictive joint kinematics 214, even if the ligament is or will be removed during the joint replacement process. A second ML model, which is trained to predict implant alignment, may provide predictions of implanted kinematics, taking into account that certain ligaments may be removed during implantation. The predictive implant alignment may provide a suggestion of an implant, and its implanted alignment, to replicate the predictive joint kinematics 214 from the first ML model. The second ML model may, therefore, take into account the removal of ligaments during the replacement procedure when providing a predicted implant alignment. In other words, an objective of implant selection and alignment/orientation through the use of the second ML model is to replicate healthy joint kinematics with all ligaments intact as determined through the first ML model although when the selected implant is implanted in accordance with the suggested alignment and orientation one or more ligaments may be sacrificed.

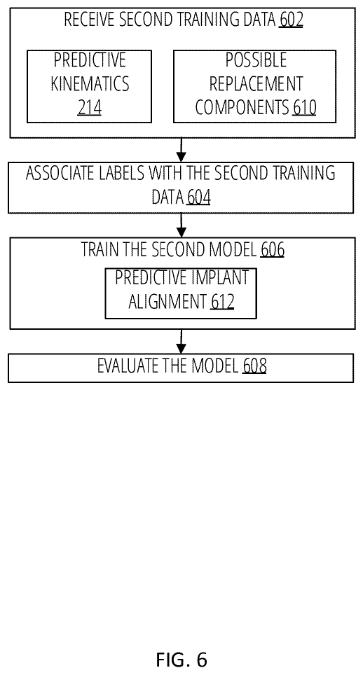

FIG. 6 illustrates an example method of training a second ML model. Training the second ML model may include receiving second training data 602, associating labels with the second training data 604, training the second model 606, and evaluating the model 210.

Receiving the second training data 602 may include, for example, receiving the predictive kinematics 214 generated by the first ML model. The second training data may also include possible replacement components 610. Possible replacement components 610 may include possible implants to be used, e.g., implanted, during a surgical technique, such as a joint replacement. For example, the possible replacement components may be a femoral component, tibial component, and/or patellar component for a knee replacement, a femoral component and acetabular component for a hip replacement, a humeral component and a glenoid component for shoulder replacement, etc. The possible replacement components may be of different sizes, types, etc.

According to some examples, the second ML model may receive the second training data 602 from storage system 130, the first ML model, server computing device 124, client computing device 128, etc. For example, the storage system 130 may receive and store the predictive kinematics 214 from the first ML model. In some examples, the storage system 130 may receive information related to the possible replacement components 610. The predictive kinematics 214 and/or possible replacement components 610 may be updated in storage system 130 as the first ML model is updated, as new implant designs are released, etc.

According to some examples, training the model may include associating labels with the second training data 602. The labels associated with the second training data 602 may be different than the labels associated with the first training data 202 used to train the first ML model. For example, a first set of labels may be associated with the first training data 202 and a second set of labels may be associated with the second training data 602. According to some examples, certain ligament labels may be removed during training of the second ML model to reflect a given procedure. For example, in a cruciate-retaining procedure, the ACL is removed. In such an example, when training the ML model to provide predictive implant alignment for a cruciate-retaining procedure, labels relating to the ACL may be removed to reflect the removal of the ACL. In another example, such as a poster-stabilized procedure, both the ACL and PCL may be removed. When training the ML model to provide predictive implant alignment for the posterior-stabilized procedure, labels relating to the ACL and PCL may be removed.

A reviewer may attach a second set of labels to the second training data 602. For example, the predicted predictive kinematics 214 and/or possible replacement components 610 may by be associated with at least one label of the second set of labels. The second set of labels may include alignment labels relating to an alignment of the possible tibial, femoral, and/or patellar components. Alignment labels may include, for example, orientation of the components to achieve the predicted kinematics, alignment of the possible components to achieve the predicted kinematics, etc.

In some examples, the second training data may be associated with the second set of labels without human review or intervention. For example, the predicted kinematics and/or possible replacement components may be automatically associated with a second label. In some examples, the predicted kinematics and/or possible replacement components may already include associated second labels at the time the second training data is received. In examples where the predicted kinematics and/or possible replacement components already include associated second labels, associating the labels with the predicted kinematics and/or possible replacement components may include confirming the associated labels and/or associating additional labels with the predicted kinematics and/or possible replacement components.

At block 606, the second model may be trained. The second ML model may be trained using the predictive kinematics 214, possible replacement components 610, and associated second labels 604 to provide a predictive implant alignment based on the respective second labels. The predictive implant alignment 612 may be, for example, predictive implanted kinematics, a suggested replacement component(s), an alignment of the replacement component(s), and/or an orientation of the replacement component(s). According to some examples, the predictive implant alignment 612 may determine a suggested replacement component(s), alignment of the replacement component(s), and/or orientation of the replacement component(s) based on a determination of when the ligaments in a joint are lax or taut such that the determined replacement component(s) and/or orientation of the replacement component(s) reproduces a ligament strain and/or elongation throughout a range of motion (“ROM”). The reproduced ligament strain and/or elongation may be determined based on a healthy joint. As an example, if the joint is a knee, the training data 602 used may include pre-operative patient imaging data. In such instances, the pre-operative patient imaging data may be reference data for the main ligaments of the knee, e.g., ACL, PCL, MCL, LCL, and the patellar tendon, indicating a laxity or tautness over a range of motion for each of the aforementioned soft tissues.

Using the knee as an example, the predictive implant alignment may include a suggested replacement component, which may be a femoral component, a tibial component, and/or patellar. The predictive implant alignment may, additionally or alternatively, include an orientation of the femoral component, an orientation of the tibial component, an orientation of the patellar component, an alignment of the femoral component with respective to the tibial component and/or patellar component, an alignment of a femoral, tibial, or patellar component with the tibia or femur, etc. According to some examples, the predictive implant alignment may include a varus-valgus alignment and internal-external rotational alignment of the suggested femoral and tibial components. In another example, the predictive implant alignment may include a posterior slope orientation of the suggested tibial component relative to the tibia. In a further example, the predictive implant alignment includes mechanical alignment of femoral and tibial component with the mechanical axis of the femur and tibia, respectively.

The second ML model can repeat the training 608 multiple times, until meeting one or more stopping criteria, such as a maximum number of training iterations. In some examples, the stopping criteria may define a minimum improvement between training iterations. Other stopping criteria may, in some examples, be based on computing resources allocated for training, a total amount of training time, etc.

The second ML model may be evaluated 608. The ML model may be evaluated based on the type of joint, the predictive kinematics, the possible replacement components, etc. For example, if the ML model performed poorly in one or more of predicting kinematics for a specific type of joint, predictive kinematics, possible replacement components, etc., the second training data may be adjusted to create more effective training data.

According to some examples a disease prediction tool may be used in conjunction with the second model. The disease prediction tool can identify the diseased state of the bony anatomy and predict the progression of disease. For any patient model, the disease prediction tool provides a b- and c-score representing the magnitude of disease progression. The b- and c-scores can be increased and decreased to generate a new bone model with increasing and decreasing signs of disease. Since the disease prediction tool provides a 3D bone model, the second model uses the bony diseased model as an input to predict kinematics according to the morphology.

In some examples, the disease prediction tool may provide a bony diseased model of the joint versus time. The bony diseased model may provide an indication of how the kinematics of the joint changes over time as the bony disease progresses. In some examples, the bony diseased model may provide an indication of the joint kinematics pre-morbid and post-morbid, e.g., based on the corresponding disease state. The pre-morbid state corresponds to a healthy representation of the bones of the joint, e.g., without osteophytes or pie crusting that would affect the prediction of the joint kinematics. The post-morbid states corresponds to the state of the bones of the joint after the onset of disease. In some examples, the post-morbid state corresponds to the shape of the bones of the joint in the arthritic condition with osteophytes. The change in joint kinematics may be used to determine an optimized implant alignment.

According to some examples, the body diseased model may be provided as input into the second ML model. For example, the second ML model may receive, as input, the body diseased model and provide, as output, predicted joint kinematics. The bony diseased model provided as input into the second ML model may be the pre-or post-morbid state of the bones within the joint.

In some examples, the disease prediction tool may be used on post-morbid bone to determine, or predict, a pre-morbid state of the bones. The predicted pre-morbid state of the bones of the joint may be provided as input, or used as training data, to the second ML model. Additionally or alternatively, the disease prediction tool may be executed on pre-morbid bone to determine, or predict, a post-morbid state of the bones. The predicted post-morbid state of the bones of the joint may be provided as input, or used as training data, to the second ML model.

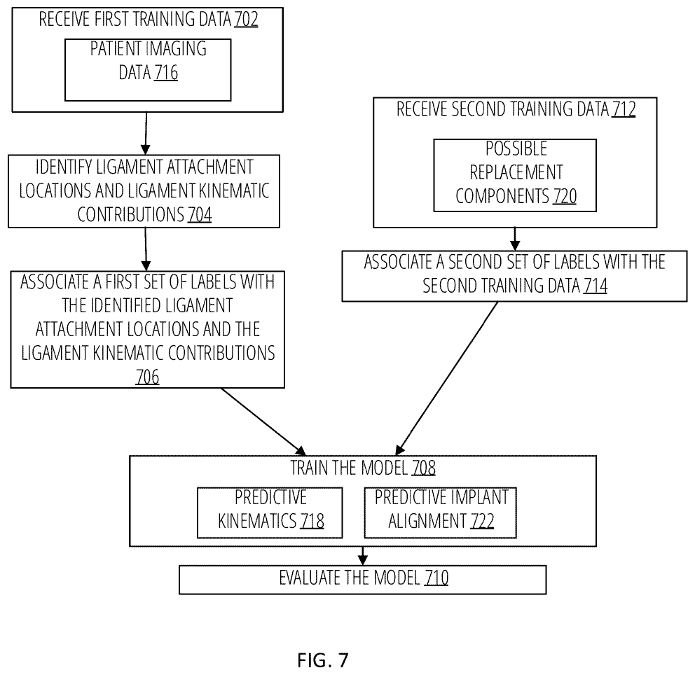

FIG. 7 is an example method for training a model. The method shown in FIG. 7 may be substantially similar to the methods described with respect to FIG. 2 and FIG. 6. However, FIG. 7 trains a single model to provide predictive kinematics and predictive implant alignment, rather than training two separate models. For example, the model may be trained by receiving first training data 702 and second training data 712. The first training data may be, for example, patient imaging data 716, and the second training data may be, for example, possible replacement components 720. Based on the patient imaging data 716, ligament attachment locations and ligament kinematic contributions may be identified 704. Training the model may include associating a first set of labels 706 with the identified ligament attachment locations and the ligament kinematic contributions and a second set of labels 714 with a second set of labels.

At block 708, the model may be trained. The ML model may be trained to predict joint kinematics 718 and implant alignment 722. According to some examples, training the ML model to predict implant alignment 722 may include predicting the joint kinematics 718 and using the predictive joint kinematics 718 to predict implant alignment 722. The predictive implant alignment may include, for example, predictive implanted kinematics. The ML model can repeat the training 708 multiple times.

Similar to the first ML model discussed with respect to FIG. 2 and the second ML model discussed with respect to FIG. 6, the ML model of FIG. 7 may be evaluated 710. The ML model may be evaluated based on the type of joint, the predictive joint kinematics, the possible replacement components, the predictive implant alignment, etc.

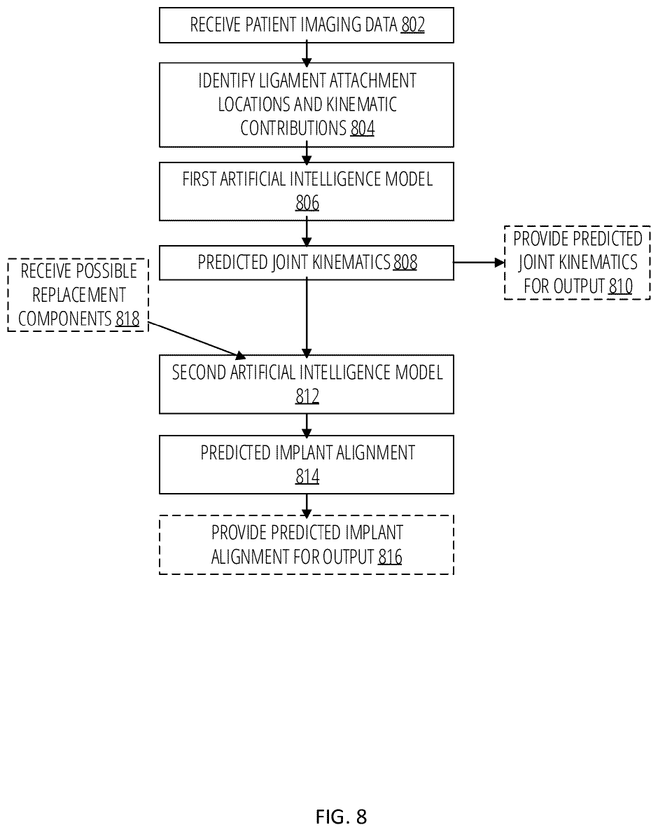

FIG. 8 illustrates an example method of applying the AI models of FIG. 2 and FIG. 6. The Al models may, in some examples, be ML models. For example, the first AI model may be used to predict joint kinematics based on patient specific imaging data and the output of the first AI model may be used to predict implant alignment for the patient.

When executing the first AI model to predict joint kinematics, the system may receive patient imaging data 802. The patient imaging data may be, for example, x-rays, CT scans, MRI scans, etc. of a joint of a specific patient seeking treatment. For example, the imaging data may be one or more images of the knee, shoulder, elbow, ankle, wrist, etc. of the patient. The imaging data may be 2-D or 3-D images. According to some examples, the imaging data may be 2-D images that are compiled, or co-registered, to create a 3-D representation of the joint.

Using the knee as an example, the patient imaging data may be images that capture each of the main cruciate and collateral ligaments. The cruciate and collateral ligaments may correspond to the “4-bar linkage”, e.g., the PCT, ACL, MCL, and LCL. In some examples, the images may capture the patellar tendon in a taut state. According to some examples, the images may be taken in a plurality of poses, such as various degrees of flexion. The degrees of flexion may include, for example, 0, 45, 60, 90 degrees, etc.

The system may use the received patient imaging data to identify ligament attachment locations and kinematic contributions 804. Ligament attachment locations may be, for example, locations on the bone(s) of the joint where the ligaments for the joint are attached. In some examples, the ligament attachment locations may be identified based on the density of a centroid or patient anatomical feature of a ligament attachment zone. For example, the ligament attachment locations may be within a ligament attachment zone. The ligament attachment zone may be a sphere, or other three-dimensional shape. The centroid having the highest density may be identified for each zone. The centroid may be, according to some examples, the ligament attachment location. Each ligament may provide its own contribution to the overall kinematics of a particular joint. The ligament kinematic contribution may include, for example, the force vector magnitudes and orientations carried by the ligaments. According to some examples, the ligament kinematic contributions may correspond to the relative influence each ligament's length as compared to the overall objective function. For example, the ACL length may have a greater contribution as compared to the PCL length when in extension. In such an example, the weight applied to the length of the ACL may be greater than the weight applied to the length of the PCL. For example, if the ACL length is determined to have twice the contribution of the PCL length when in extension, the ACL weight may be 2 while the PCL weight may be 1. According to some examples, the contributions and, therefore, respective weights applied to the contributions may change as a function of flexion. While the system is shown as identifying ligament attachment locations and kinematic contributions 804 before applying the ML model, the ML model may be used to identify ligament attachment locations and kinematic contributions 804.

According to some examples, the system may use the received patient imaging data to determine the volumetric strain, or stiffness, metric for each ligament. The volumetric strain metric may be determined based on a ratio of the change in volume to the original volume. The change in volume may be, for example, the change in volume of the ligament between one pose of the ligament in a taut position to another pose of the ligament in a taut position. The pose may be, for example, the position and orientation of the ligament. The volumetric strain metric may be determined for the entire ligament, specific bundles and/or fibers within the ligament, or the like.

The AI model is trained to predict joint kinematics using 3D bony anatomy and ligament contribution. The ligament contribution may be determined based on, at least in part, the volumetric strain metric. For example, each patient is assigned a unique ligament weight factor based on the underlying bone density of the ligament attachment and/or the volumetric ligament strain. Once the volumetric strain metric is determined, and the patients associated ligament weight factor, the relative contribution of the ligaments within the joint may be determined and provided to the AI model.

The identified ligament attachment locations and kinematics contributions 804 may be input into the first AI model 806. According to some examples, the volumetric strain metrics may be provided as input into the first AI model 806. The first AI model may output predictive joint kinematics 808. The predictive joint kinematics 808 may be, for example, a prediction of the movement between the bones in the joint with respect to one another when the bones are undegenerated, or healthy. Using the knee as an example, the predictive knee kinematics may include a prediction of the movement of the femur with respect to the tibia when the femur and tibia are undegenerated. According to some examples, the predictive joint kinematics 808 may include a target for implanting the possible replacement components. Continuing with the knee, the predictive joint kinematics 808 may include a target for implanting at least one of a tibial component, a femoral component, and/or a patellar component.

According to some examples, the system may provide the predictive joint kinematics 808 for output 810. Providing the predicted joint kinematics 808 for output 810 may include, for example, providing the predictive joint kinematics 808 for output on a client computing device 128. Providing the predictive joint kinematics 808 for output on a client computing device 128 may include, for example, providing the predicted joint kinematics 808 for display on a display screen, monitor, etc. such that a user, e.g., a physician, can view and/or interact with the predictive joint kinematics 808.

According to some examples, after the first AI model predicts the joint kinematics, the system may receive information related to possible replacement components 818. In some examples, the system may store the information related to the possible replacement components in memory of a server computing device 124 and/or in storage system 130. The system may, therefore, receive, or recall, the information related to possible replacement components when applying the second AI model. In some examples, the system may have previously received the information related to possible replacement components. Accordingly, receiving information related to possible replacement components 818 is optional.

The second AI model may receive, as input, the predictive joint kinematics 808 and the possible replacement components 818. According to some examples, the second AI model may receive, as input, the volumetric strain metrics for the ligaments. The second AI model may output predicted implant alignment 814. The predicted implant alignment may include, for example, one or more suggested replacement components, a cut location, a possible surgical plan, a suggested orientation for implanting the one or more replacement components, a suggested alignment of the one or more replacement components, etc. According to some examples, the predicted implant alignment may be provided for output 816. Providing the predicted implant alignment for output 816 may include, for example, providing the predicted implant alignment for display on a display screen, monitor, etc. of a client computing device 128. Alternatively, the predicted implant alignment may be communicated to an intraoperative computing device to help guide the surgeon in a navigated procedure or to perform a robotic procedure.

According to some examples, the predicted implant alignment 814 may include an implant type and position that, when taken through a ROM as part of a joint, performs so that the soft tissue of the joint substantially corresponds to the ligament strain or elongation pattern exhibited in a pre-disease or pre-deterioration condition of the joint. In some examples, the second Al model may account for any intra-operative ligament removal that may occur when implanting the implant. The predicted implant alignment may be based on the patient anatomy. In some examples, the predicted implant alignment may minimize strain between pre-operative and intra-operative conditions.

In some examples, the predicted implant alignment 814 may be applied to a disease bone model generated by a disease prediction model. For example, the predicted implant alignment 814 may be provided as input into the disease prediction model. The disease prediction model may be trained to predict kinematics using the bony anatomy, healthy or diseased, or implant geometry. The bony anatomy, e.g., bone geometry, is used during pre-resection and the implant geometry is used during intra-operative planning. The optimal implant alignment may correspond to the implant position that minimizes the differences between the pre-operative and intra-operative joint kinematics and ligament volumetric strain. The disease prediction model may generate a prediction of the joint kinematics over time for the corresponding disease state. The prediction may allow for the kinematics of the joint to be understood over time as a result of the bone disease progression. For example, an optimized implant alignment may be determined based on one or more of predicted disease progression in combination. pre-morbid kinematics, patient anatomy, minimization of ligament strain, or the like.

While the method shown in FIG. 8 is based on using a first and second AI model, the method may be performed using a single AI model. For example, the system may receive patient imaging data 802 and possible replacement components 818. The system, or the AI model, may identify ligament attachment locations and kinematic contributions 804. The AI model may output predictive joint kinematics 808 and/or predicted implant alignment 814. The predictive joint kinematics 808 and/or predicted implant alignment 814 may be provided for output 810, 816.

According to some examples, the predictive joint kinematics 808 and/or predicted implant alignment 814 may be used as training data for the first and second machine learning models. For example, after implementing the first machine learning model 806 to predict joint kinematics 808 for a specific patient, the predictive joint kinematics 808 output by the first machine learning model may be used to evaluate the first machine learning model and/or be used as training data for the first machine learning model. Similarly, after implementing the second machine learning mode 812 to predict implant alignment 814, the predicted implant alignment 814 output by the second machine learning model may be used to evaluate the second machine learning model and/or be used as subsequent training data for the second machine learning model.

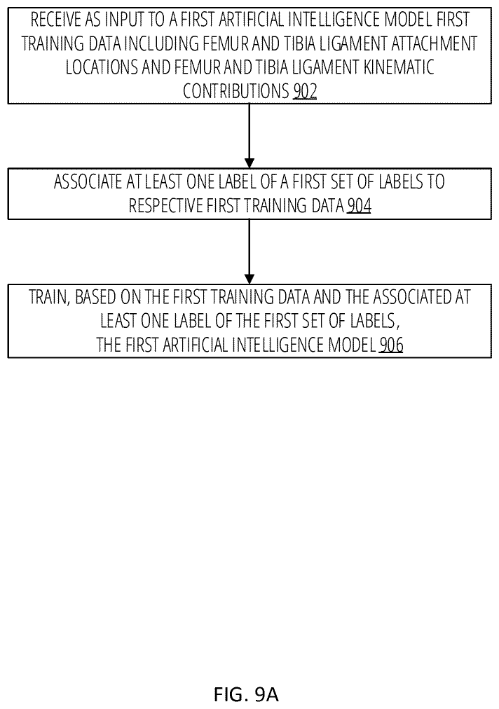

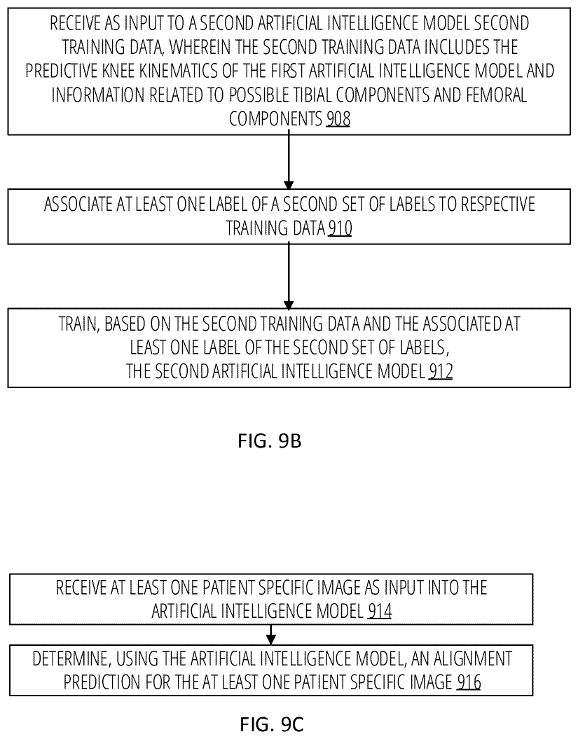

FIG. 9A illustrates an example method for training an Al model to predict joint kinematics. The AI model may, in some examples, be a ML model. The following operations do not have to be performed in the precise order described below. Rather, various operations can be handled in a different order or simultaneously, and operations may be added or omitted.

In block 902, first training data may be received as input into a first AI model. The first training data may include, for example, femur and tibia ligament attachment locations and femur and tibia ligament kinematic contributions. According to some examples, the first training data may further include tibial, femoral, and/or patellar geometry data.

In block 904, at least one label of a first set of labels may be associated with respective first training data. The first set of labels may include ligament length labels relating to a minimum total ligament length for a knee to move through a range of motion while maintaining a constant joint space between a femur and tibia.

In block 906, the first AI model may be trained based on the first training data and the associated at least one label of the first set of labels. The first AI model may be trained to provide predicted knee kinematics. The predictive knee kinematics may include, for example, a predictive of a movement of a femur and a tibia with respect to one another when the femur and the tibia are undegenerated, or healthy. In some examples, the predictive knee kinematics may correspond to a target for implanting at least one of a tibial component, a femoral component, and a patellar component.

FIG. 9B illustrates an example method for training an Al model to predict implant alignment. The Al model may, in some examples, be a ML model. The following operations do not have to be performed in the precise order described below. Rather, various operations can be handled in a different order or simultaneously, and operations may be added or omitted.

In block 908, second training data may be received as input to a second AI model. The second training data may include, for example, the predictive knee kinematics of the first AI model and information related to possible tibial components, femoral components, and patellar components.

In block 910, at least one label of a second set of labels may be associated with respective second training data. The second set of labels may include, for example, alignment labels relating to an alignment of the possible tibial, femoral, and patellar components.

In block 912, the second AI model may be trained based on the second training data and the associated at least one label of the second set of labels. The second AI model may be trained to provide predictive implant alignment for at least one of the femoral components, the tibial components, or the patellar components. The predictive implant alignment may include at least one of a suggested femoral component, a suggested tibial component, a suggested patellar component, an orientation of the femoral component, an orientation of the tibial component, an orientation of the patellar component, or an alignment of the femoral, tibial, and/or patellar component. According to some examples, the predictive implant alignment may include at least one of a varus-valgus alignment and internal-external rotational alignment of the suggested femoral and tibial components. In another example, the predictive implant alignment may include a posterior slope orientation of the suggested tibial component.

FIG. 9C illustrates an example method for applying, or executing, the AI models to predict joint kinematics and/or implant alignment. The following operations do not have to be performed in the precise order described below. Rather, various operations can be handled in a different order or simultaneously, and operations may be added or omitted.

In block 914, the AI model(s) may receive at least one patient specific image as input. According to some examples, the at least one patient specific image may be received as input by the second ML model. The at least one patient specific image may include, for example, an x-ray image, CT scan, MRI scan, etc.

In block 916, the AI model(s), such as the second AI model, may determine a predictive implant alignment for the at least one patient specific image. The alignment prediction for the at least one patient specific image may include, for example, at least one of a suggested femoral component, a suggested tibial component, a suggested patellar component, an orientation of the femoral component, an orientation of the tibial component, an orientation of the patellar component, or an alignment of the femoral, tibial, and/or patellar component. According to some examples, the alignment prediction may, additionally or alternatively, include a suggested cut location or a suggested surgical plan.

Although the invention herein has been described with reference to particular examples, it is to be understood that these examples are merely illustrative of the principles and applications of the present invention. It is therefore to be understood that numerous modifications may be made to the illustrative examples and that other arrangements may be devised without departing from the spirit and scope of the present invention as defined by the appended claims.

1. A method, comprising:
receiving as input to a first artificial intelligence model, by one or more processors, first training data including femur and tibia ligament attachment locations and femur and tibia ligament kinematic contributions;
associating, by the one or more processors, at least one label of a first set of labels to respective first training data, wherein the first set of labels includes ligament length labels relating to a minimum total ligament length for a knee to move through a range of motion while maintaining a constant joint space between a femur and tibia; and
training, by the one or more processors based on the first training data and the associated at least one label of the first set of labels, the first artificial intelligence model, wherein the first artificial intelligence model outputs predictive knee kinematics.

2. The method of claim 1, wherein the predictive knee kinematics include a prediction of a movement of the femur and a tibia move with respect to one another when the femur and the tibia are undegenerated.

3. The method of claim 1, wherein the predictive knee kinematics correspond to a target for implanting at least one of a tibial component, a femoral component, or a patellar component.

4. The method of claim 1, wherein the first training data further includes tibial, femoral, and patellar geometry data.

5. The method of claim 1, further comprising:
receiving as input to a second artificial intelligence model, by the one or more processors, second training data, wherein the second training data includes the predictive knee kinematics of the first artificial intelligence model and information related to possible tibial components, femoral components, and patellar components;
associating, by the one or more processors, at least one label of a second set of labels to respective second training data, wherein the second set of labels includes alignment labels relating to an alignment of the possible tibial, femoral components, and patellar components; and
training, by the one or more processors based on the second training data and the associated at least one label of the second set of labels, the second artificial intelligence model, wherein the second artificial intelligence model outputs predictive implant alignment for at least one of the possible femoral components, the possible tibial components, or the possible patellar components.

6. The method of claim 5, wherein the predictive implant alignment includes at least one of a suggested femoral component, a suggested tibial component, a suggested patellar component, an orientation of the femoral component, an orientation of the tibial component, an orientation of the patellar component, or an alignment of the femoral, tibial, and patellar components.

7. The method of claim 6, wherein the predictive implant alignment includes at least one of a varus-valgus alignment, internal-external rotational alignment, medial-lateral position, anterior-posterior position, or flexion-extension position of the suggested femoral and tibial components.

8. The method of claim 7, wherein the predictive implant alignment further includes a posterior slope orientation of the suggested tibial component.

9. The method of claims 5, further comprising executing the second artificial intelligence model to identify at least one of an implant or an alignment of an implant, the executing comprising:
receiving at least one patient specific image as input into the second artificial intelligence model; and
determining, using the second artificial intelligence model, at least one of an implant prediction or an implant alignment prediction for the at least one patient specific image.

10. The method of claim 9, wherein the alignment prediction for the at least one patient specific image includes at least one of a suggested femoral component, a suggested tibial component, a suggested patellar component, an orientation of the femoral component, an orientation of the tibial component, an orientation of the patellar component, or an alignment of the femoral, tibial, and patellar components.

11. The method of claim 9, wherein the at least one patient specific image includes a CT scan.

12. The method of claim 9, further comprising:
determining, by the one or more processors based on the at least one patient specific image, a volumetric strain metric for a ligament in the knee; and
providing, as input into the second artificial intelligence model, the volumetric strain metric.

13. The method of claim 12, wherein determining the volumetric strain metric comprises:
comparing, by the one or more processors, a first volume of the ligament in a first pose to a second volume of the ligament in a second pose, wherein the ligament is in a taut condition in the first and second poses; and
determining, by the one or more processors based on the comparison, a change in volume between the first pose and the second pose, wherein the change in volume corresponds to the volumetric strain metric.

14. A system comprising:
one or more processors, the one or more processors configured to:
receive as input to a first artificial intelligence model first training data including femur and tibia ligament attachment locations and femur and tibia ligament kinematic contributions;
associate at least one label of a first set of labels to respective first training data, wherein the first set of labels includes ligament length labels relating to a minimum total ligament length for a knee to move through a range of motion while maintaining a constant joint space between a femur and tibia; and
train, based on the first training data and the associated at least one label of the first set of labels, the first artificial intelligence model, wherein the first artificial intelligence model outputs predictive knee kinematics.

15. The system of claim 14, wherein the predictive knee kinematics include a prediction of a movement of the femur and a tibia move with respect to one another when the femur and the tibia are undegenerated.

16. The system of claim 14, wherein the predictive knee kinematics correspond to a target for implanting at least one of a tibial component or a femoral component.

17. The system of claim 14, wherein the first training data further includes tibial and femoral geometry data.

18. The system of claim 14, wherein the one or more processors are further configured to:
receive as input to a second artificial intelligence model second training data, wherein the second training data includes the predictive knee kinematics of the first artificial intelligence model and information related to possible tibial components, femoral components, and patellar components;
associate at least one label of a second set of labels to respective second training data, wherein the second set of labels includes alignment labels relating to an alignment of the possible tibial, femoral, and patellar components; and
train, based on the second training data and the associated at least one label of the second set of labels, the second artificial intelligence model, wherein the second artificial intelligence model outputs predictive implant alignment for at least one of the possible femoral components, the possible tibial components, or the possible patellar components.

19. The system of claim 18, wherein the predictive implant alignment includes at least one of a suggested femoral component, a suggested tibial component, a suggested patellar component, an orientation of the femoral component, an orientation of the tibial component, an orientation of the patellar component, or an alignment of the femoral, tibial, and patellar components.

20. A non-transitory computer-readable storage medium, the computer-readable storage medium including instructions that when executed by one or more processors, cause the one or more processors to:
receive as input to a first artificial intelligence model first training data including femur and tibia ligament attachment locations and femur and tibia ligament kinematic contributions;
associate at least one label of a first set of labels to respective first training data, wherein the first set of labels includes ligament length labels relating to a minimum total ligament length for a knee to move through a range of motion while maintaining a constant joint space between a femur and tibia; and
train, based on the first training data and the associated at least one label of the first set of labels, the first artificial intelligence model, wherein the first artificial intelligence model outputs predictive knee kinematics.
